(19) United States
(12) Patent Application Publication
(10) Pub. No.: US 2025/0288452 A1
(43) Pub. Date: Sep. 18, 2025

(54) REVERSIBLE FEMALE URINARY DEVICE

(71) Applicant: HYGIE CANADA INC., Brossard (CA)

(72) Inventors: Eric Tanguay, Brossard (CA); Isabelle Tremblay, Lac-Sergent (CA); Marianne Lancelot, Québec (CA); Dominic Lemelin, Québec (CA); Edith Martin, Québec (CA); Éric Pelletier, St-Félix-de-Kingsey (CA); Jimmy Riopel-Thibault, Saint-Paul-de-l'Ile-aux-Noix (CA); Pierre-Michel Lahaye, Brossard (CA)

(73) Assignee: HYGIE CANADA INC., Brossard, QC (CA)

(21) Appl. No.: 18/861,319

(22) PCT Filed: Apr. 28, 2023
(86) PCT No.: PCT/CA2023/050575

§ 371 Date: Oct. 29, 2024

Related U.S. Application Data
(60) Provisional application No. 63/363,882, filed on Apr. 29, 2022.

(51) Int. Cl. A61F 5/455

(52) U.S. Cl. CPC A61F 5/455

(57) ABSTRACT

The present disclosure concerns a reversible female urinary device comprising: a urine-collecting housing defining a hollow space and having at least one urine-collecting inlet opening into the hollow space and having an inlet height; and at least one sealing rim at least partially surrounding the urine-collecting inlet, wherein, considered along the inlet height, said sealing rim forms first and second opposed user-contacting curved end portions, thereby defining at least two distinct usage configurations of the urinary device. It also concerns a corresponding urinary assembly.

### CROSS-REFERENCE TO RELATED APPLICATIONS

The present application is a national stage application filed under 35 U.S.C. § 371 of PCT Application No. PCT/CA2023/050575, filed Apr. 28, 2023, which claims priority from U.S. provisional patent application No. 63/363,882, filed on Apr. 29, 2022, the disclosures of each of which are hereby incorporated by reference in their entirety.

### BACKGROUND OF THE DISCLOSURE

### 1. Field of the Disclosure

The technical field relates to a urinary device, and more particularly to a non-invasive (or external) female urinary device that is configurable into a plurality of usage configurations.

### 2. Description of Related Art

The direction of female urine flow requires females to be substantially seated during urination. The inability and/or difficulty to direct urine flow might be problematic for example when attempting to use a bed pan or similar device if bedridden, as it is often not possible to properly arrange the bed pan at the right location and/or inclination angle to catch urine stream, or to predict where the urine stream may initially be directed.

It is therefore known to provide female urination devices for enabling a female to channel, direct and collect their urine flow. However, such devices might be shaped and dimensioned for a specific morphology and can thus not be comfortable or suitable for anyone.

In view of the above, there is a need for a female urinary device which would be able to overcome or at least minimize some of the above-discussed prior art concerns.

### SUMMARY OF THE DISCLOSURE

It is therefore an aim of the present disclosure to at least partially address the above-mentioned issues.

According to a general aspect, there is provided a reversible female urinary device comprising: a urine-collecting housing defining a hollow space and having at least one urine-collecting inlet opening into the hollow space and having an inlet height; and at least one sealing rim at least partially surrounding the urine-collecting inlet, wherein, considered along the inlet height, said sealing rim forms first and second opposed user-contacting curved end portions, thereby defining at least two distinct usage configurations of the urinary device.

According to another general aspect, there is provided a reversible female urinary device having a longitudinal direction and comprising: a body at least partially delimiting a hollow space and having first and second opposed longitudinal ends, at least one urine-collecting inlet being formed at one of the first and second longitudinal ends, the urine-collecting inlet opening into the hollow space and having an inlet height, and a sealing flange extending outward and away from the urine-collecting inlet, wherein, considered along the inlet height, said sealing flange forms first and second opposed user-contacting curved portions, thereby defining at least two possible usage configurations of the urinary device.

According to another general aspect, there is provided a reversible female urinary device defining a longitudinal direction and comprising: a body at least partially delimiting a hollow space and having at least a first longitudinal end opening into the hollow space and having a first height, and a first sealing rim at least partially surrounding the first longitudinal end, wherein, considered along the first height, said first sealing rim forms first and second opposed user-contacting curved portions, thereby defining at least two possible usage configurations of the urinary device.

According to another general aspect, there is provided a reversible female urinary device defining a longitudinal direction and comprising: a substantially tubular wall at least partially delimiting a hollow space and having first and second opposed longitudinal ends opening into the hollow space, and first and second sealing rims at least partially surrounding respectively the first and second longitudinal ends to form opposed first and second user-contacting surfaces, thereby defining at least two possible usage configurations.

According to another general aspect, there is provided a reversible female urinary assembly, comprising: a reversible female urinary device according to the present disclosure, and a disposable bag fittable into the hollow space and securable to the urine-collecting housing.

According to another general aspect, there is provided a reversible female urinary assembly, comprising: a reversible female urinary device according to the present disclosure, and a disposable bag fittable into the hollow space and securable to the body.

### BRIEF DESCRIPTION OF THE DRAWINGS

FIG. 1 is a first side perspective view of a reversible female urinary device in accordance with a first embodiment;

FIG. 2 is a second side perspective view of the urinary device of FIG. 1;

FIG. 3 is a top perspective view of the urinary device of FIG. 1;

FIG. 4 is a first side elevational view of the urinary device of FIG. 1, the urinary device being arranged in a first usage configuration;

FIG. 5 is a second side elevational view of the urinary device of FIG. 1, the urinary device being arranged in a second usage configuration and being upside down with respect to the first usage configuration;

FIG. 6 is a first end elevational view of the urinary device of FIG. 1, the device comprising a first sealing rim;

FIG. 7 is a second end elevational view of the urinary device of FIG. 1, the device comprising a second sealing rim;

FIG. 8 is a schematic flattened representation of an ellipse defined by the first sealing rim of the urinary device of FIG. 1;

FIG. 9 is a schematic flattened representation of an ellipse defined by the second sealing rim of the urinary device of FIG. 1;

FIG. 10 is a schematic representation of curvatures defined by the first sealing rim of the urinary device of FIG. 1, projected onto a plane of symmetry thereof;

FIG. 11 is a schematic representation of curvatures defined by the second sealing rim of the urinary device of FIG. 1, projected onto the plane of symmetry thereof;

FIG. 12 is a front top perspective view of a reversible female urinary device in accordance with a second embodiment;

FIG. 13 is a front bottom perspective view of the urinary device of FIG. 12;

FIG. 14 is a rear top perspective view of the reversible female urinary device of FIG. 12;

FIG. 15 is a side elevational view of the reversible female urinary device of FIG. 12;

FIG. 16 is a cross-sectional view of the reversible female urinary device of FIG. 12, taken along cross-section lines 16-16 of FIG. 15; and

FIG. 17 is front elevational view of the reversible urinary device of FIG. 12.

### DETAILED DESCRIPTION

In the following description, the same numerical references refer to similar elements. Furthermore, for the sake of simplicity and clarity, namely so as to not unduly burden the figures with several references numbers, not all figures contain references to all the components and features, and references to some components and features may be found in only one figure, and components and features of the present disclosure which are illustrated in other figures can be easily inferred therefrom. The embodiments, geometrical configurations, materials mentioned and/or dimensions shown in the figures are optional and are given for exemplification purposes only.

Moreover, it will be appreciated that positional descriptions such as “above”, “below”, “forward”, “rearward”, “left”, “right” and the like should, unless otherwise indicated, be taken in the context of the figures only and should not be considered limiting. Moreover, the figures are meant to be illustrative of certain characteristics of the reversible urinary device and are not necessarily to scale.

To provide a more concise description, some of the quantitative expressions given herein may be qualified with the term “about”. It is understood that whether the term “about” is used explicitly or not, every quantity given herein is meant to refer to an actual given value, and it is also meant to refer to the approximation to such given value that would reasonably be inferred based on the ordinary skill in the art, including approximations due to the experimental and/or measurement conditions for such given value.

In the following description, an embodiment is an example or implementation. The various appearances of “one embodiment”, “an embodiment” or “some embodiments” do not necessarily all refer to the same embodiments. Although various features may be described in the context of a single embodiment, the features may also be provided separately or in any suitable combination. Conversely, although the disclosure may be described herein in the context of separate embodiments for clarity, it may also be implemented in a single embodiment. Reference in the specification to “some embodiments”, “an embodiment”, “one embodiment” or “other embodiments” means that a particular feature, structure, or characteristic described in connection with the embodiments is included in at least some embodiments, but not necessarily all embodiments.

It is to be understood that the phraseology and terminology employed herein is not to be construed as limiting and are for descriptive purpose only. The principles and uses of the teachings of the present disclosure may be better understood with reference to the accompanying description, figures and examples. It is to be understood that the details set forth herein do not construe a limitation to an application of the disclosure.

Furthermore, it is to be understood that the disclosure can be carried out or practiced in various ways and that the disclosure can be implemented in embodiments other than the ones outlined in the description above. It is to be understood that the terms “including”, “comprising”, and grammatical variants thereof do not preclude the addition of one or more components, features, steps, or integers or groups thereof and that the terms are to be construed as specifying components, features, steps or integers. If the specification or claims refer to “an additional” element, that does not preclude there being more than one of the additional element. It is to be understood that where the claims or specification refer to “a” or “an” element, such reference should not be construed as meaning that there is only one of that element. It is to be understood that where the specification states that a component, feature, structure, or characteristic “may”, “might”, “can” or “could” be included, that particular component, feature, structure, or characteristic is not required to be included.

The descriptions, examples, methods and materials presented in the claims and the specification are not to be construed as limiting but rather as illustrative only. Meanings of technical and scientific terms used herein are to be commonly understood as by one of ordinary skill in the art to which the disclosure belongs, unless otherwise defined. It will be appreciated that the methods described herein may be performed in the described order, or in any suitable order.

The present disclosure concerns a reversible female urinary device 100, 1100 comprising a urine-collecting housing 200, 1200 (or body 200, 1200) defining a hollow space 210, 1210 and having at least one urine-collecting inlet 220, 1220 (or first longitudinal end 220, 1220) opening into the hollow space and having an inlet height H1; and at least one sealing rim 300, 1300 at least partially surrounding the urine-collecting inlet, wherein, considered along the inlet height H1, said sealing rim forms first and second opposed user-contacting curved end portions 310, 320, 1310, 1320, thereby defining at least two possible usage configurations of the urinary device 100, 1100.

The reversible female urinary device 100, 1100 of the present disclosure is a non-invasive (or external) urinary device, i.e., is configured to be worn externally by a female user, around the user's urethral orifice, to collect urine excreted through the urethral orifice. As used herein, the term “external” when used to describe the urinary device 100, 1100 refers to an assembly which is adapted to be used without inserting components inside the female's urethra.

As detailed below, a preferred version of the non-invasive female urinary device 100, 1100 is constructed of plastic, typically through an injection molding process. As a result, the device is light-weight, low-cost and easily cleaned. While certain parts of the device may be referred to individually, as detailed below, it is important to note that in a possible embodiment, the entire device would be made in one piece for reasons of economy and quality. It could also be conceived a urinary device that would be made of a plurality of components—for instance at least partially made of distinct materials—that would be secured together.

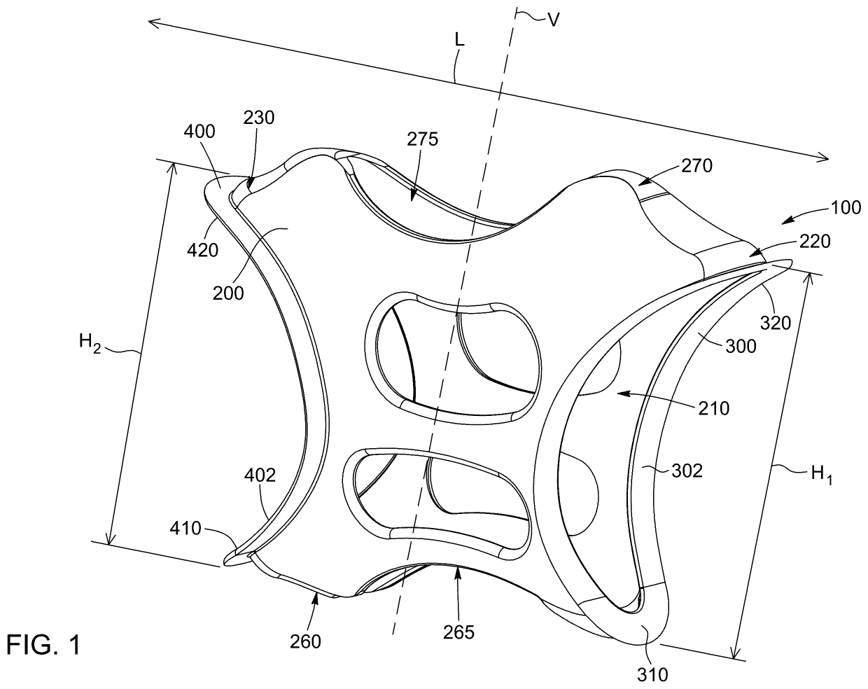

Referring now to the drawings, and more particularly to FIGS. 1 to 11, there is shown a first embodiment of the reversible female urinary device 100 (or multi-configuration female urinary device 100). The urinary device 100 defines a longitudinal direction L and comprises a body 200—or substantially tubular wall 200, or urine-collecting housing 200—at least partially delimiting a hollow space 210 (or bag-receiving cavity 210) and having at least a first longitudinal end 220—or first longitudinal mouth 220 or urine-collecting inlet 220—opening into the hollow space 210 and having a first height H1 or first inlet height H1, and a first sealing rim 300 or first flange 300 at least partially surrounding the first longitudinal end 220, wherein, considered along the first height H1, the first sealing rim 300 forms first and second opposed user-contacting curved portions 310, 320, thereby defining at least two possible usage configurations of the female urinary device 100. In other words, and as detailed below, the female urinary device 100 is reversible at least upon rotation of the device 100 about the longitudinal direction L thereof, so as to arrange either the first user-contacting curved portion 310 or the second user-contacting curved portion 320 proximate a surface supporting the urinary device 100 and bordering at least partially a lower portion of the genitals of the user. In other words, in a first usage configuration, the first and second opposed user-contacting curved portions 310, 320 form respectively posterior and anterior ends of the first sealing rim 300 according to the anatomical orientation of the urinary device 100 when worn by a user. When worn by a user, the posterior end is positioned posterior of the user's urethral orifice while the anterior end is positioned anterior of the user's urethral orifice. In a second usage configuration, the second and first opposed user-contacting curved portions 320, 310 form respectively the posterior and anterior ends of the first sealing rim 300.

As detailed below, in the first embodiment of the urinary device 100, the body 200 or substantially tubular wall 200 further comprises a second longitudinal end 230—or second longitudinal mouth 230—opening into the hollow space 210, opposed to the first longitudinal end 220, wherein the urinary device 100 further comprises a second sealing rim 400 surrounding at least partially the second longitudinal end 230 to form a second user-contacting surface 402 longitudinally opposed to a first user-contacting surfaces 302 formed by the first sealing rim 300. In other words, the second sealing rim 400 defines additional possible usage configurations of the urinary device. In yet other words, the female urinary device 100 is also reversible upon rotation of the device about an axis V (FIG. 1) substantially perpendicular to the longitudinal direction L of the urinary device 100. As detailed below, in a similar manner to the first sealing rim 300, the second sealing rim 400 forms, considered along a second height H2 of the second sealing rim 400, first and second opposed user-contacting curved portions 410, 420, thereby defining at least two additional possible usage configurations of the female urinary device 100. It is thus understood that in a third usage configuration, the first and second opposed user-contacting curved portions 410, 420 form respectively the posterior and anterior ends of the second sealing rim 400, while in a fourth usage configuration, the second and first opposed user-contacting curved portions 420, 410 form respectively the posterior and anterior ends of the second sealing rim 400.

In other words, the reversible urinary device 100 according to the present disclosure is configurable into four different usage configurations.

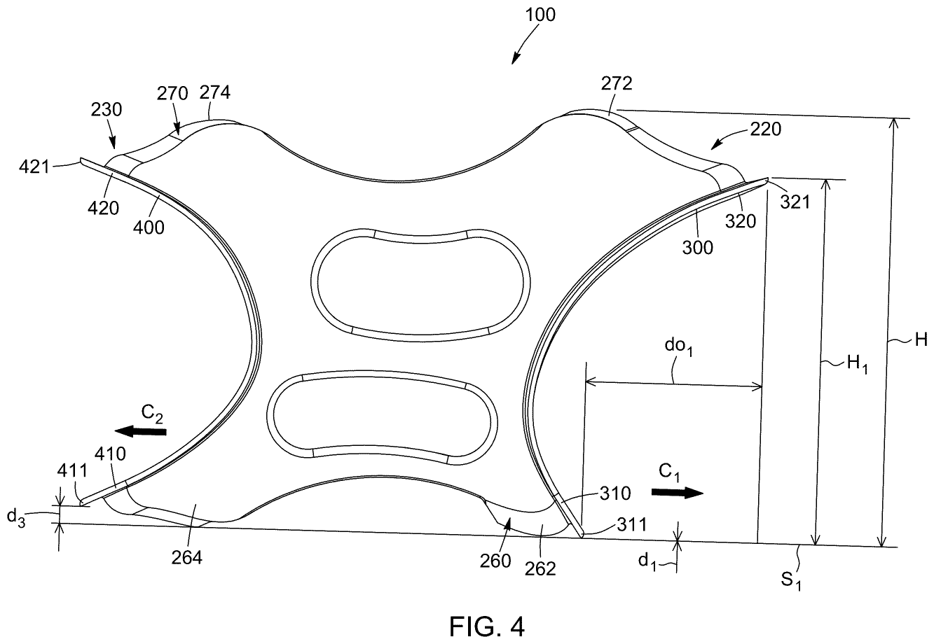

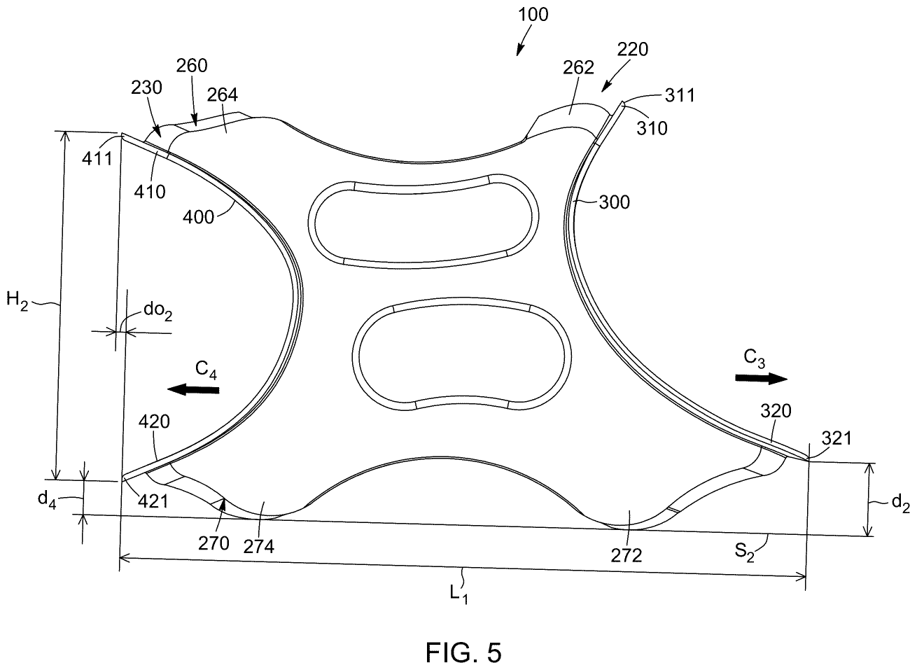

As best shown in FIGS. 4 and 5, considered in a plane comprising the longitudinal direction and the first and second heights H1, H2, a profile of the first and second sealing rims 300, 400 is substantially C-shaped.

### Body

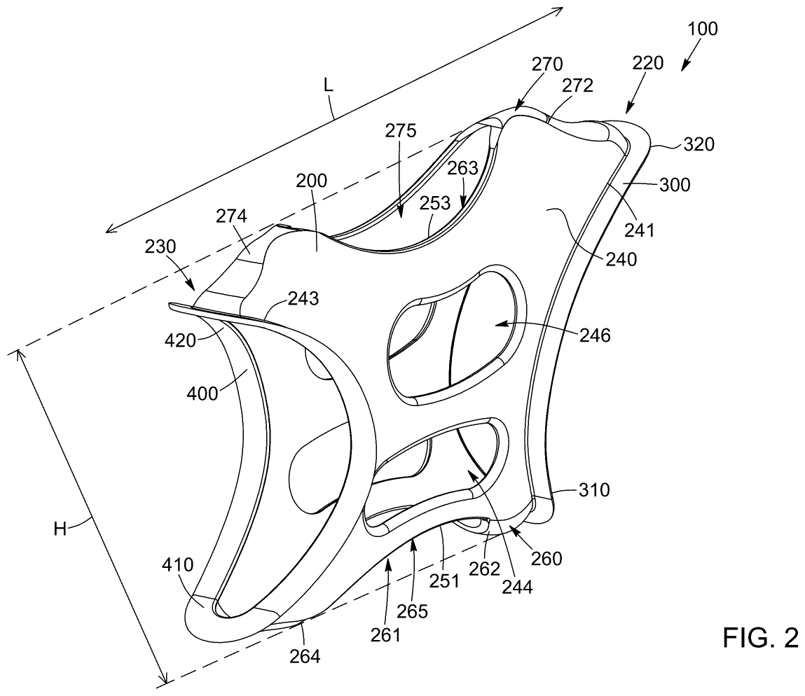

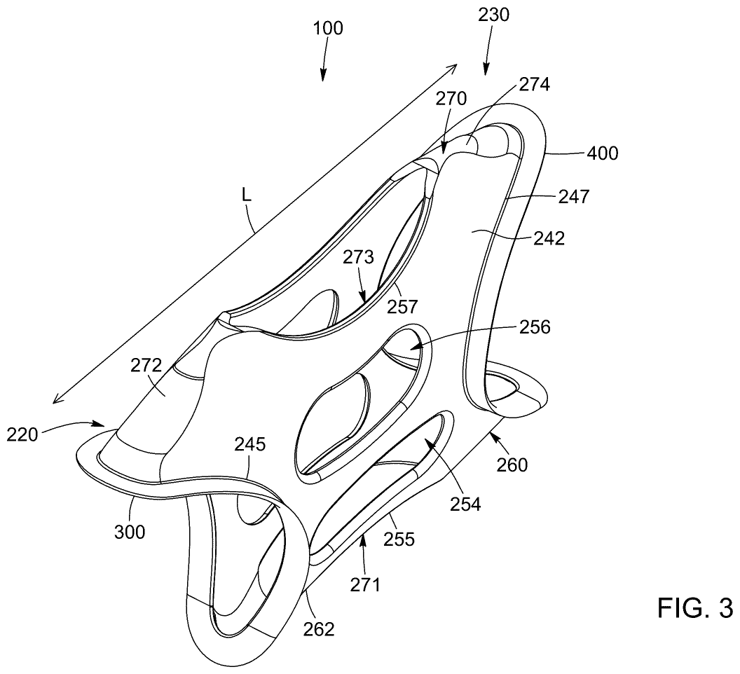

Referring to FIGS. 2 and 3, in the embodiment shown, the body 200 is substantially tubular in shape (i.e., is at least partially formed of the substantially tubular wall delimiting at least partially the hollow space 210). In the embodiment shown, the body 200 comprises first and second side walls 240, 242 extending along the longitudinal direction L and along the axis V, substantially parallel to each other, and spaced apart from each other. Each of the first and second side walls 240, 242 extends between the first and second longitudinal ends 220, 230; inner faces of the first and second opposed side walls 240, 242 at least partially delimit the hollow space 210.

Considered along the longitudinal direction L, each of the first and second side walls 240, 242 comprises first and second opposed mouth-forming edges 241, 243, 245, 247 (FIGS. 2 and 3) forming or delimiting at least partially the first and second longitudinal ends 210, 230. As represented, concavities are formed in the mouth-forming edges of the first and second side walls 240, 242, for the first and second longitudinal ends to better conform to the morphology of the user.

Considered along a height H (FIG. 4) of the body 200, each of the first and second side walls 240, 242 further comprises first and second opposed gripping edges 251, 253, 255, 257, which extend along the longitudinal direction L of the urinary device 100. The height H is understood as being the greater dimension in a vertical dimension, when the urinary device is supported on a horizontal plane.

In the embodiment shown, two lateral holes—or gripping openings—244, 246, 254, 256, for instance having a substantially oblong and oval shape, are formed in each of the first and second side walls 240, 242 and open into the hollow space 210. The gripping openings are shaped and dimensioned for a hand of the user to be at least partially inserted therein, in order to ease the handling of the urinary device 100. In the embodiment shown, the lateral holes 244, 246 formed in the first side wall 240 are substantially in register with the lateral holes 254, 256 formed in the second side wall 242. For instance, and as detailed below, the lateral holes are shaped and dimensioned to allow an introduction of a bag in the hollow space 210 and/or to remove a bag therefrom. The lateral holes might also be used by the user to adjust the position of the urinary device between their legs. In the embodiment shown, a periphery of the gripping openings forms a convexity substantially following a profile of the closest edge of the corresponding side wall. In the embodiment shown, the periphery of the griping openings forms a concavity, opposed to the convexity when considered along the height of the urinary device, directed toward a center of the corresponding sidewall.

Considered along the height H of the body 200, the body 200 further comprises first and second opposed connecting portions 260, 270 extending between the first and second side walls 240, 242 and connecting at least partially the first and second gripping edges 251, 253, 255, 257 of the first and second side walls 240, 242, so as to connect at least partially the first and second side walls 240, 242. Inner surfaces of the first and second connecting portions 260, 270 at least partially delimit the hollow space 210 of the body 200.

In the embodiment shown, each of the first and second connecting portions 260, 270 comprises at least first and second gripping parts 262, 264, 272, 274, spaced apart from each other and forming apexes of the first and second connecting portions. The first and second gripping parts 262, 264, 272, 274 connect together portions (longitudinal end portions thereof, in the embodiment shown) of the corresponding first and second gripping edges 251, 253, 255, 257 of the first and second side walls 240, 242.

In the embodiment shown, the first and second gripping parts 262, 264, 272, 274 are proximate respectively the first and second longitudinal ends 220, 230. It is thus understood that central portions of each of the first and second gripping edges 251, 253, 255, 257 of the first and second side walls 240, 242 are spaced apart from each other and disconnected from each other. In other words, as best shown in FIGS. 1 and 2, bag-introducing openings 265, 275 are formed in the first and second connecting portions 260, 270, at least partially delimited, considered along a width W1 of the urinary device 100, by the first and second gripping edges of the first and second side walls 240, 242 and, considered along the longitudinal direction L of the urinary device, by the corresponding first and second gripping parts 262, 264, 272, 274 of the first and second connecting portions 260, 270. In other words, the first and second gripping parts 262, 264, 272, 274 of the first and second connecting portions 260, 270 are spaced apart from each other and define—or at least partially delimit—longitudinal bag-introducing openings 265, 275 therebetween, which open into the hollow space. In the embodiment shown, the bag-introducing openings 265, 275 are substantially in register with each other, oval in shape and form, considered in a plane comprising the height H of the urinary device 100, concavities in the first and second gripping edges 251, 253, 255, 257 of the first and second side walls 240, 242.

As best shown in FIGS. 4 and 5, the first and second gripping parts 262, 264, 272, 274 of the first and second connecting portions 260, 270 form first and second convexities, directed outwardly from the hollow space 210 of the body 200, which are shaped and dimensioned to prevent flow of urine to flow back outside the hollow cavity 210 when the user is seated or lying.

As mentioned above, concavities 261, 263, 271, 273 are formed in the gripping edges 251, 253, 255, 257 of the first and second side walls 240, 242 between the corresponding first and second gripping parts 262, 264, 272, 274 of the first and second connecting portions 260, 270, which delimit at least partially the above-mentioned bag-introducing openings 265, 275.

In the embodiment shown, the body 200 has a first plane of symmetry parallel to the height H and the longitudinal direction L of the body 200 and extending between the first and second side walls 240, 242.

It is appreciated that the shape and the configuration of the body 200 (or urine-collecting housing 200), for instance the shape and the configuration of the first and second side walls and the first and second connecting portions thereof, as well as the shape, the configuration and the number of the gripping parts, the bag-introducing openings and the lateral holes thereof can vary from the embodiment shown.

### First and Second Sealing Rims (First Embodiment)

In the first embodiment shown, the first and second sealing rims 300, 400 are formed integral with the tubular body 200, respectively at the first and second longitudinal ends 220, 230 thereof. In other words, the first and second sealing rims 300, 400 extend outward and away respectively from the first and second longitudinal ends of the urinary device 100; each of the first and second sealing rims 300, 400 provides a surface area sufficient to form an adhesive bond with the user's skin to hold the urinary device 100 in place. It could also be conceived a urinary device wherein the first and second sealing rims would be assembled or mounted or connected to the tubular wall 200 or body 200 after being separately manufactured, for instance in a material different from the one forming at least partially the body 200. In other words, each of the first and second user-contacting surfaces formed respectively by the first and second sealing rims 300, 400 forms an interface surface that is configured to be adhered to the user's body around the user's urethral orifice.

In other words, in the embodiment shown the body 200 bends (outwardly in the embodiment shown) at at least one of the first and second longitudinal ends 220, 230 (at both of them, in the embodiment shown) in a pointed or rounded way to form the first and second sealing rims 300, 400.

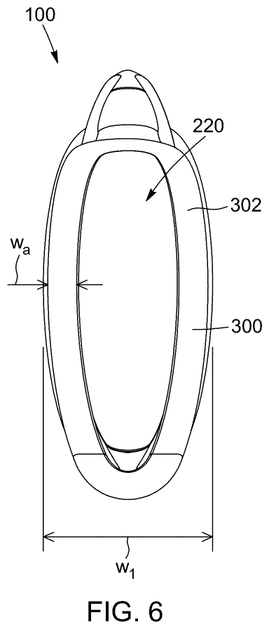

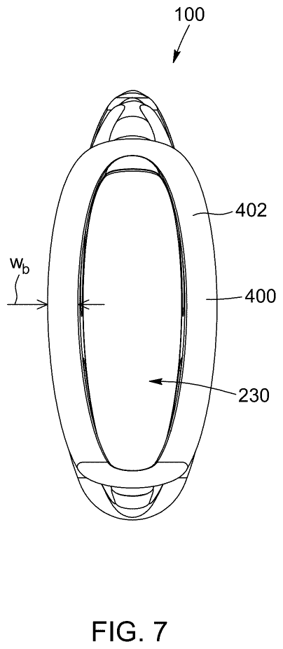

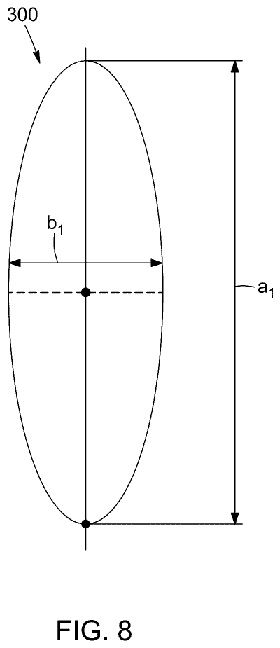

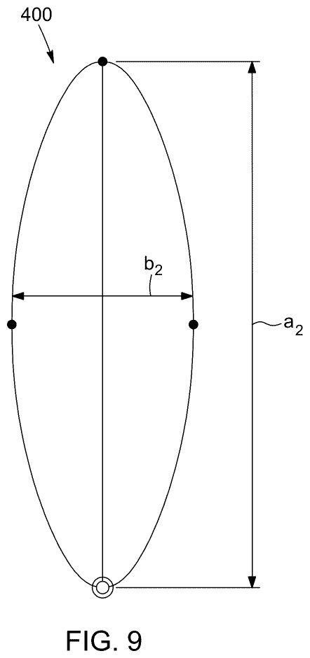

As best shown in FIGS. 6 and 7, a transversal cross-section (i.e., considered in a plane substantially perpendicular to the longitudinal direction L for instance between the first gripping parts 262, 272 of the first and second connecting portions 260, 270, on the one hand, and the second gripping parts 272, 274 of the first and second connecting portions 260, 270, on the other hand) of the tubular wall/body 200 is substantially oval. Moreover, a transversal cross-section of the first and second longitudinal ends and of the first and second sealing rims is substantially oval. As best shown in FIGS. 4, 8 and 9, the first and second sealing rims 300, 400, for instance the first and second user-contacting surfaces 302, 402 thereof, both define a folded or curved ellipse. In other words, if the first and second user-contacting surfaces 302, 402 were flattened onto a plane, they would both form an ellipse, as represented in FIGS. 8 and 9.

For instance, the ellipse formed by the flattened first user-contacting surface 302, as represented in FIG. 8, has a major axis a1 comprised between about 100 mm and about 200 mm, for instance comprised between about 120 mm and about 180 mm, for instance comprised between about 140 mm and about 160 mm, for instance of about 150 mm. The ellipse formed by the flattened first user-contacting surface 302 has a minor axis b1 comprised between about 20 mm and about 80 mm, for instance comprised between about 30 mm and about 70 mm, for instance comprised between about 40 mm and about 60 mm, for instance of about 50 mm. For instance, the ellipse formed by the flattened first user-contacting surface 302 has an eccentricity greater than about 0.5, for instance greater than about 0.7, for instance greater than about 0.8, for instance greater than about 0.9, for instance greater than about 0.95. For instance, an inner perimeter of the flattened first user-contacting surface 302 bordering the first opening 220 is comprised between about 200 mm and about 600 mm, for instance comprised between about 300 mm and about 500 mm, for instance comprised between about 350 mm and about 450 mm, for instance of about 410 mm.

For instance, the ellipse formed by the flattened second user-contacting surface 402, as represented in FIG. 9, has a major axis a2 comprised between about 100 mm and about 220 mm, for instance comprised between about 120 mm and about 200 mm, for instance comprised between about 140 mm and about 180 mm, for instance of about 150 mm. The ellipse formed by the flattened second user-contacting surface 400 has a minor axis b2 comprised between about 25 mm and about 85 mm, for instance comprised between about 35 mm and about 75 mm, for instance comprised between about 50 mm and about 60 mm, for instance of about 55 mm. For instance, the ellipse formed by the flattened second user-contacting surface 402 has an eccentricity greater than about 0.5, for instance greater than about 0.7, for instance greater than about 0.8, for instance greater than about 0.9, for instance greater than about 0.95. For instance, an inner perimeter of the flattened second user-contacting surface 402 bordering the second opening 230 is comprised between about 200 mm and about 600 mm, for instance comprised between about 300 mm and about 500 mm, for instance comprised between about 350 mm and about 450 mm, for instance comprised between about 400 mm and about 450 mm, for instance of about 430 mm.

In the embodiment shown, the first and second sealing rims 300, 400 surround respectively an entirety of a periphery of the first and second longitudinal ends 220, 230. In other words, the first and second sealing rims 300, 400 are substantially annular in shape and surround the corresponding one of the first and second longitudinal ends 220, 230. In the embodiment shown, the first and second sealing rims are curved along the height and along a width of the corresponding one of the first and second longitudinal ends 220, 230. In other words, in the embodiment shown, each of the first and second sealing rims comprises first and second end wall portions (forming at least partially the first and second opposed user-contacting curved portions) and first and second sidewalls, spaced apart from each other and extending along the height of the corresponding one of the first and second longitudinal ends).

Considered in a plane comprising the longitudinal direction L and the height H1, H2 of the corresponding one of the first and second longitudinal ends 220, 230, as best shown in FIGS. 4 and 5, the first and second opposed user-contacting curved portions 310, 320, 410, 420 have different radius of curvature. It could also be conceived a urinary device wherein only some of the user-contacting curved portions would have different radius of curvature.

Each of the first and second opposed user-contacting curved portions 310, 320, 410, 420 comprises a tip 311, 321, 411, 421 (or distal end or end wall portions), which defines a tip distance d1, d2, d3, d4 with support-contacting surfaces S1, S2 defined by the closest one of the first and second connecting portions 260, 270, as represented in FIGS. 4 and 5. In other words, each of the first and second connecting portions 260, 270 defines a support-contacting surface S1, S2 connecting distal ends—or apexes—of the corresponding ones of the first and second gripping parts 262, 264, 272, 274: the first support-contacting surface S1 connects the distal ends of the first and second gripping parts 262, 264 of the first connecting portion 260 and the second support-contacting surface S2 connects the distal ends of the first and second gripping parts 272, 274 of the second connecting portion 270. The tips 311, 411 of the first user-contacting curved portions 310, 410 of the first and second sealing rims 300, 400 are respectively at distances d1, d3 of the first support-contacting surface S1 (FIG. 4), whereas the tips 321, 421 of the second user-contacting curved portions 320, 420 of the first and second sealing rims 300, 400 are respectively at distances d2, d4 of the second support-contacting surface S2 (FIG. 5).

In the embodiment shown, the first sealing rim 300 is shaped and dimensioned so that the tip 311 of the first user-contacting end portion 310 is proximate to or slightly in contact with the first support-contacting surface S1 (i.e., the tip distance d1 is substantially null). In the embodiment shown, the tip distances d1, d2, d3, d4 of the corresponding tips 311, 321, 411, 421 are different from each other. It could also be conceived a urinary device wherein only some of the tip distances would be different from each other.

The tip distance of one of the first and second user-contacting curved portions of each of the first and second sealing rims 300, 400 is different from the tip distance of the other one of the first and second user-contacting curved portions of the corresponding one of the first and second sealing rims 300, 400. In other words, the tip distances d1, d2 of the first sealing rim 300 are different from each other and the tip distances d3, d4 of the second sealing rim 400 are different from each other.

Moreover, the tip distance d1 between the tip 311 of the first user-contacting portion 310 of the first sealing rim 300 and the first support-contacting surface S1 is different from the tip distance d3 between the tip 411 of the first user-contacting portion 410 of the second sealing rim 400 and the first support-contacting surface S1 (see FIG. 4). The tip distance d2 between the tip 321 of the second user-contacting portion 320 of the first sealing rim 300 and the second support-contacting surface S2 is different from the tip distance d4 between the tip 421 of the second user-contacting portion 420 of the second sealing rim 400 and the second support-contacting surface S2 (see FIG. 5).

For instance, a tip distance between the tip of one of the first and second user-contacting portions of one of the first and second sealing rims and the corresponding support-contacting surface S1, S2 is greater than about 1%, for instance greater than about 3%, for instance greater than about 5%, for instance greater than about 10% of the height H1, H2 of the corresponding one of the first and second longitudinal ends 220, 230.

In the embodiment shown, as represented in FIGS. 4 and 5, the tip of one of the first and second user-contacting curved portions of each of the first and second sealing rims 300, 400 is longitudinally offset with respect to the tip of the other one of the first and second user-contacting curved portions of the corresponding one of the first and second sealing rims 300, 400. In other words, considered along the longitudinal direction L, the tip 311 of the first user-contacting curved portion 310 of the first sealing rim 300 is longitudinally offset (inwardly in the embodiment shown, with respect to the hollow space) with respect to the tip 321 of the second user-contacting curved portion 320 of the first sealing rim 300. A tip offset distance do1 is thus defined between the tips 311, 321. Moreover, considered along the longitudinal direction L, the tip 421 of the second user-contacting curved portion 420 of the second sealing rim 400 is longitudinally offset (inwardly in the embodiment shown, with respect to the hollow space) with respect to the tip 411 of the first user-contacting curved portion 410 of the second sealing rim 400. A tip offset distance do2 is thus defined between the tips 411, 421. For instance, the tip offset distance d02 is smaller than the tip offset distance do1. For instance, one of the tip offset distances is smaller than about 75%, for instance smaller than about 50%, for instance smaller than about 25%, for instance smaller than about 10% of the other one of the tip offset distances.

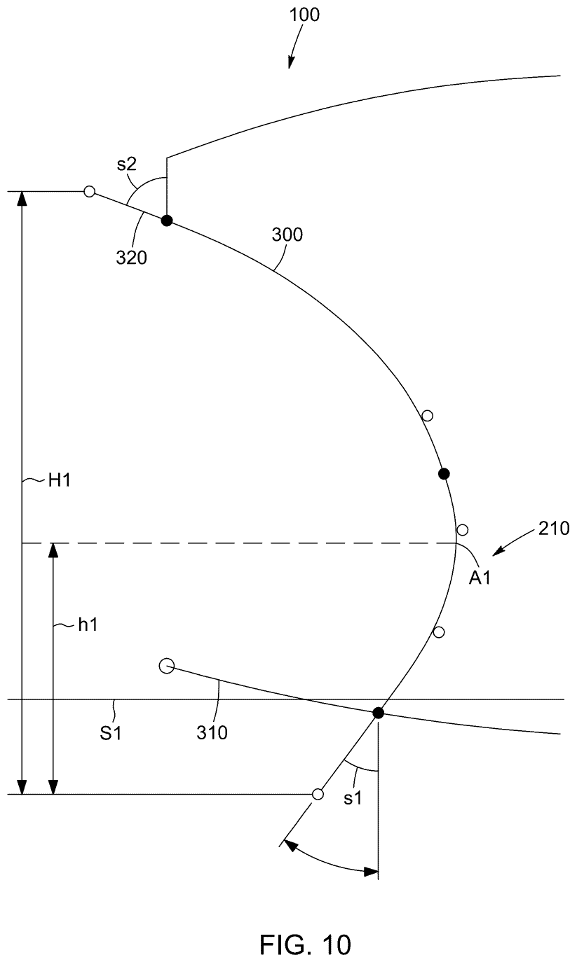

As best shown in FIG. 10, when projected onto the plane of symmetry of the urinary device 10, a curvature of the first sealing rim 300 varies along the height of the first sealing rim 300. For instance, a slope of a first angle s1 is defined by an inner side (considered with respect to the hollow space 210) of the first user-contacting curved portion 310 of the first sealing rim 300 and a direction perpendicular to the first support-contacting surface S1. For instance, the first angle s1 is comprised between about 10 degrees and about 60 degrees, for instance comprised between about 20 degrees and about 50 degrees, for instance comprised between about 30 degrees and about 40 degrees, for instance of about 36 degrees. For instance, a slope of a second angle s2 is defined by an inner side (considered with respect to the hollow space 210) of the second user-contacting curved portion 320 of the first sealing rim 300 and a direction perpendicular to the first support-contacting surface S1. For instance, the second angle s2 is comprised between about 40 degrees and about 100 degrees, for instance comprised between about 50 degrees and about 90 degrees, for instance comprised between about 60 degrees and about 80 degrees, for instance comprised between about 65 degrees and about 75 degrees, for instance of about 70 degrees.

For instance, an apex A1 of the curvature of the first sealing rim 300 (i.e., wherein a slope is null) is at a height h1 smaller than a half of the height H1 of the first longitudinal end 220. For instance, the height h1 is smaller than about 45%, for instance smaller than about 40%, of the height H1 of the first longitudinal end 220.

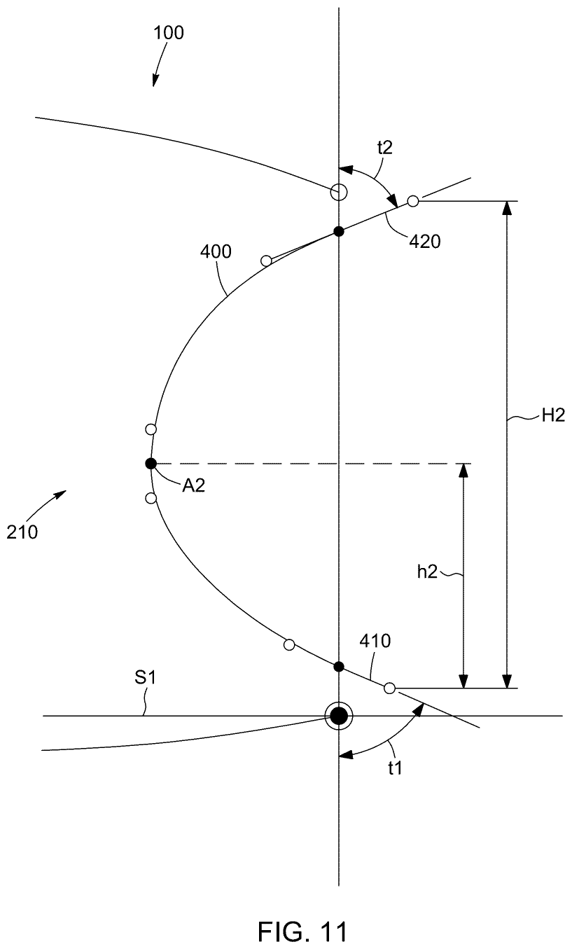

As best shown in FIG. 11, when projected onto the plane of symmetry of the urinary device 10, a curvature of the second sealing rim 400 varies along the height of the second sealing rim 400. For instance, a slope of a first angle t1 is defined by an inner side (considered with respect to the hollow space 210) of the first user-contacting curved portion 410 of the second sealing rim 400 and a direction perpendicular to the first support-contacting surface S1. For instance, the first angle t1 is comprised between about 15 degrees and about 115 degrees, for instance comprised between about 30 degrees and about 100 degrees, for instance comprised between about 50 degrees and about 80 degrees, for instance comprised between about 60 degrees and about 70 degrees, for instance of about 66 degrees. For instance, a slope of a second angle t2 is defined by an inner side (considered with respect to the hollow space 210) of the second user-contacting curved portion 420 of the second sealing rim 400 and a direction perpendicular to the first support-contacting surface S1. For instance, the second angle t2 is comprised between about 40 degrees and about 100 degrees, for instance comprised between about 50 degrees and about 90 degrees, for instance comprised between about 60 degrees and about 80 degrees, for instance comprised between about 65 degrees and about 75 degrees, for instance of about 70 degrees.

For instance, an apex A2 of the curvature of the second sealing rim 400 (i.e., wherein a slope is null) is at a height h2 smaller than a half of the height H2 of the second longitudinal end 230. For instance, the height h2 is smaller than about 50%, for instance smaller than about 47%, for instance smaller than about 45% of the height H2 of the second longitudinal end 230.

A length L1 of the urinary device 100 is defined, considered along the longitudinal direction L, between the furthest tips of the first and second sealing rims 300, 400. In the embodiment shown, the length L1 is defined between the tips 321, 421 of the second user-contacting curved portions 320, 420 of the first and second sealing rims 300, 400. For instance, the tip offset distance between the tips of the first and second user-contacting portions of one of the first and second sealing rims is greater than about 1%, for instance greater than about 3%, for instance greater than about 5%, for instance greater than about 10%, for instance greater than about 25% of the length L1 of the urinary device 100.

The length L1 of the urinary device 100 is comprised between about 10 cm and about 40 cm, for instance between about 20 cm and about 30 cm, for instance comprised between about 23 cm and about 29 cm, for instance of about 26 cm.

The heights H1, H2 of the first and second longitudinal ends 220, 230 are comprised between about 5 cm and about 40 cm, for instance comprised between about 10 cm and about 25 cm, for instance comprised between about 15 cm and about 20 cm, for instance of about 19 cm. For instance, the first and second heights H1, H2 are substantially equal.

The width W1 of the urinary device 100, as represented in FIG. 6, is comprised between about 2 cm and about 12 cm, for instance comprised between about 4 cm and about 10 cm, for instance comprised between about 6 cm and about 8 cm, for instance comprised between about 6 cm and about 7 cm, for instance comprised between about 6.2 cm and about 6.8 cm.

As mentioned above, the first and second sealing rims 300, 400 define first and second user-contacting surfaces 302, 402 at least partially delimiting the first and second longitudinal ends 220, 230 opening into the hollow space 210. The first and second user-contacting surfaces 302, 402 extend locally transversally, for instance substantially perpendicularly, to outer surfaces of the body 200 (to edges of the body 200 forming the periphery of the corresponding one of the first and second longitudinal ends).

As best shown in FIGS. 6 and 7, the first and second user-contacting surfaces have a substantially constant width wa, wb along substantially an entirety of the periphery of the corresponding one of the first and second longitudinal ends 220, 230. For instance the width wa, wb of the first and second user-contacting surfaces 302, 402 is greater than about 1%, for instance greater than about 5%, for instance greater than about 10%, for instance greater than about 15% of the width W1 of the urinary device 100. For instance, the width wa, wb is comprised between about 3 mm and about 20 mm, for instance comprised between about 7 mm and about 16 mm, for instance comprised between about 9 mm and about 14 mm, for instance comprised between about 11 mm and about 12 mm, for instance of about 11.5 mm.

For instance, the first and second sealing rims are at least partially formed in a material having softness and elasticity properties, so as to ensure a usage comfort and a substantially fluid-tight contact with the user. For instance, the first and second sealing rims are at least partially formed in a material being more flexible and resilient than a material in which the body is at least partially formed.

It is thus understood that the urinary device 100 is outwardly bent at the first and second ends 220, 230 of the body 200, to form the first and second sealing rims 300, 400. Various designs could be applied to the first and second sealing rims in order to increase comfort when in contact with or applied near the genitals of the user.

It is appreciated that the shape and the configuration of the first and second sealing rims 300, 400, for instance the radius of curvature, shape, configuration and relative positions of the first and second opposed user-contacting curved portions thereof, can vary from the embodiment shown. It is also appreciated that the shape and the configuration of the reversible female urinary device is not limited to the embodiment represented in FIGS. 1 to 11, wherein first and second sealing rims at least partially surround respectively the first and second longitudinal ends of the urine-collecting housing 200.

### Single Sealing Rim (Second Embodiment)

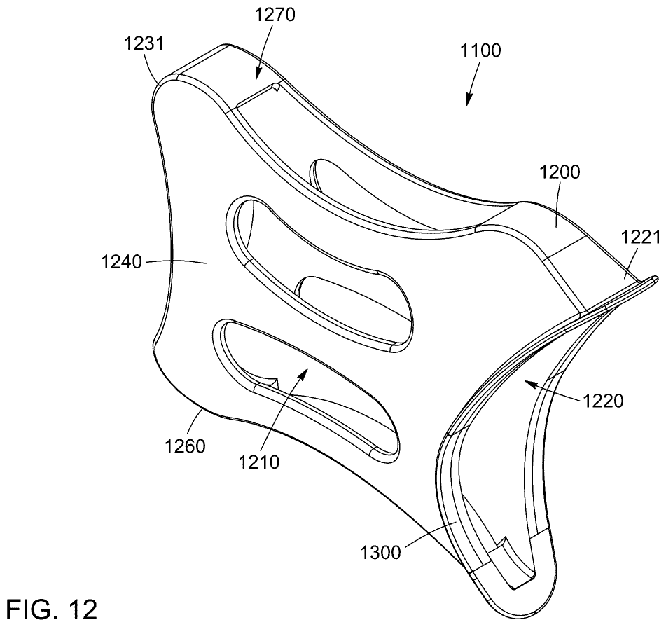

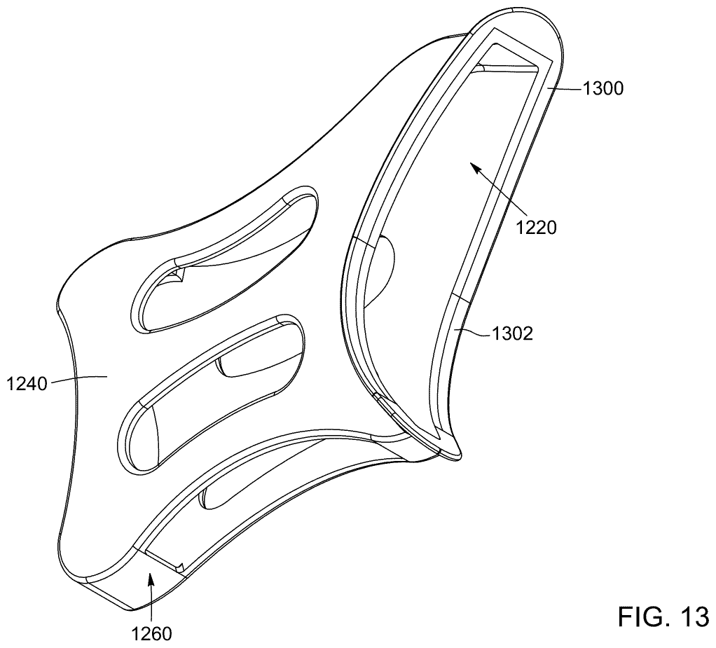

Referring now to FIGS. 12 to 17, these is shown a second possible embodiment of the reversible female urinary device 1100. In a similar manner to the first embodiment, the urinary device 1100 comprises a urine-collecting housing 1200 defining a hollow space 1210 and having at least one urine-collecting inlet 1220 opening into the hollow space 1210 and having an inlet height H1. The urinary device 1100 further comprises a sealing rim 1300 at least partially surrounding the urine-collecting inlet 1220 and forming a user-contacting surface 1302, wherein, considered along the inlet height H1, said sealing rim 1300 forms first and second opposed user-contacting curved end portions 1310, 1320, thereby defining at least two possible usage configurations of the urinary device.

The second embodiment mainly differs from the first embodiment in that the urinary device 1100 comprises a single sealing rim 1300 at one of the first and second longitudinal ends 1221, 1231 of the body 1200.

It is understood that although various features have been described in the context of the first embodiment of the urinary device 100, the features may also be provided to the second embodiment of the urinary device 1100, and vice-versa.

In the embodiment shown, the urine-collecting inlet 1220 is formed at one of the first and second longitudinal ends 1221, 1231 of the urine-collecting housing 1200.

The sealing rim 1300 (or single sealing rim 1300, in the embodiment shown) surrounds substantially an entirety of a periphery of the urine-collecting inlet 1220 and comprises first and second end wall portions, forming respectively the first and second opposed user-contacting curved end portions 1310, 1320 and first and second opposed sidewalls, spaced apart from each other and extending along the inlet height H1 and connecting the first and second curved end portions 1310, 1320. In the embodiment shown, a continuity is formed by the first and second end wall portions and the first and second opposed sidewalls.

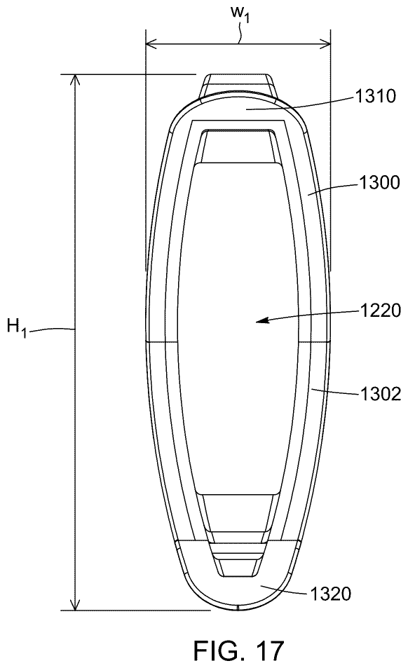

For instance, the inlet height H1 of the opening or urine-collecting inlet 1220 (as represented in FIG. 17) is comprised between about 100 mm and about 220 mm, for instance comprised between about 120 mm and about 200 mm, for instance comprised between about 140 mm and about 180 mm, for instance of about 160 mm.

For instance, an inlet width W1 of the opening or urine-collecting inlet 1220 (as represented in FIG. 17) is comprised between about 30 mm and about 60 mm, for instance comprised between about 35 mm and about 55 mm, for instance comprised between about 40 mm and about 50 mm, for instance of about 44 mm.

In the embodiment shown, a length of the periphery of the opening or urine-collecting inlet 1220 is comprised between about 200 mm and about 600 mm, for instance comprised between about 300 mm and about 500 mm, for instance comprised between about 400 mm and about 450 mm, of instance of about 420 mm.

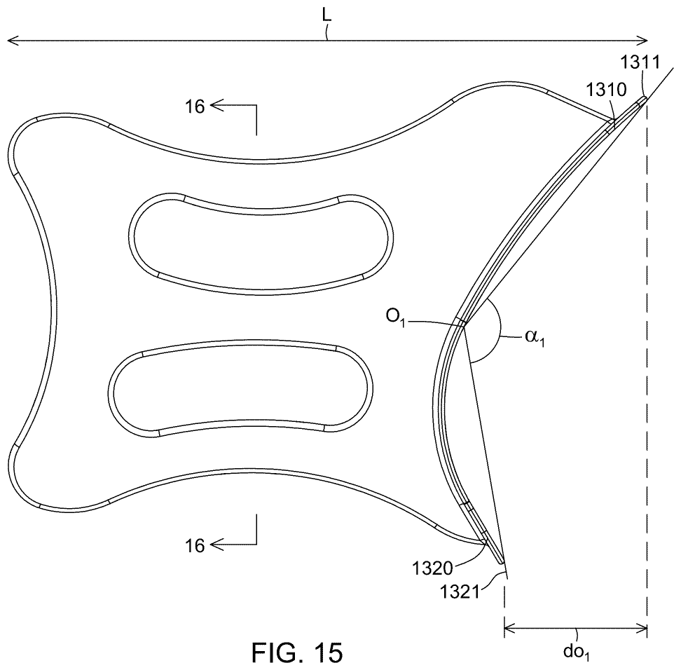

As best shown in FIG. 15, considered in a plane comprising the longitudinal direction L of the body 1200 and the inlet height H1, the first and second user-contacting curved portions 1310, 1320 have first and second radii of curvature different from each other. In other words, when projected onto the plane of symmetry of the urinary device 1100, said plane comprising the longitudinal direction L of the body 1200 and the inlet height H1, a curvature of the sealing rim 1300 varies along the height H1 of the sealing rim 1300.

As represented in FIG. 15, the sealing rim 1300 defines an opening angle α1 of the urine collecting inlet 1220. For instance, an origin O1 of the opening angle α1 is located at a half height of the inlet height H1. The opening angle α1 is defined between a first line extending from the origin O1 towards a tip 1311 of the first user-contacting curved end portion 1310, and a second line extending from the origin O1 towards a tip 1321 of the second user-contacting curved end portion 1320. For instance, the opening angle α1 is comprised between about 80 degrees and about 180 degrees, for instance comprised between about 100 degrees and about 160 degrees, for instance comprised between about 120 degrees and about 140 degrees, for instance of about 130 degrees.

In a similar manner to the first embodiment, the tips 1311, 1321 of the first and second user-contacting curved portions 1310, 1320 of the first sealing rim 1300 are longitudinally offset with respect to each other from a tip offset distance do1 (FIG. 15).

In the second embodiment, and contrary to the first embodiment, no sealing rim is provided at the second longitudinal end of the body 1200, so that the urinary device 1100 in accordance with the second embodiment is configurable into only first and second usage configurations: in the first usage configuration, the first and second opposed user-contacting curved portions 1310, 1320 form respectively posterior and anterior ends of the sealing rim 1300 according to the anatomical orientation of the urinary device 100 when worn by a user, and in the second usage configuration, the second and first opposed user-contacting curved portions 1320, 1310 form respectively the posterior and anterior ends of the sealing rim 1300.

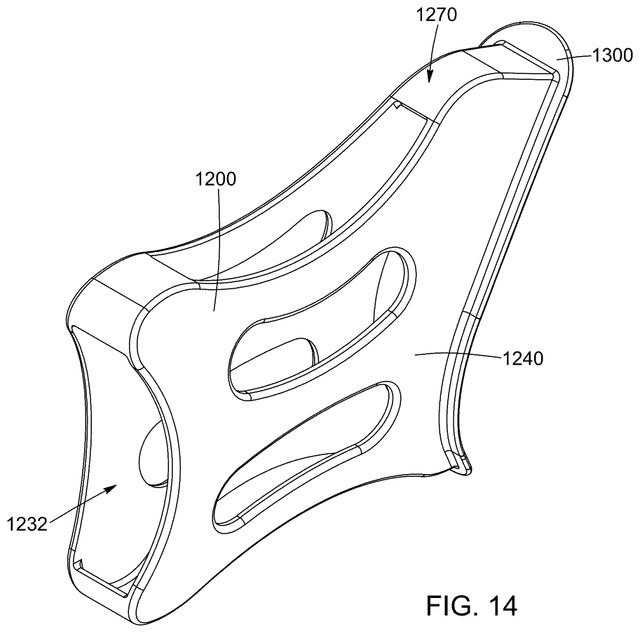

In the second embodiment, as best shown in FIG. 14, a bag-introducing opening 1232 is formed at the second longitudinal end 1231 of the body 1200 which opens into the hollow space 1210. For instance, the second longitudinal end 1231 of the body 1200 forms a handling portion shaped and dimensioned to increase a contact of the sealing rim 1300 with the user, when the urinary device 1100 is used.

In the embodiment shown, considered along the height of the body 1200, the first and second end portions of the second longitudinal end 1231 of the body 1200 are substantially rounded or curved.

In a similar manner to the first embodiment, the urine-collecting housing or body 1200 comprises first and second side walls 1240, 1242 spaced apart from each other, and first and second connecting portions 1260, 1270 extending at least partially between the first and second side walls 1240, 1242. At least one gripping opening is formed in at least one of the first and second side walls. For instance, gripping openings are formed in the first and second side walls, substantially in register with each other. For instance, in the embodiment shown, first and second gripping openings are formed in each of the first and second side walls 1240, 1242. For instance, a periphery of the gripping opening forms a convexity substantially following a profile of the closest edge of the corresponding side wall.

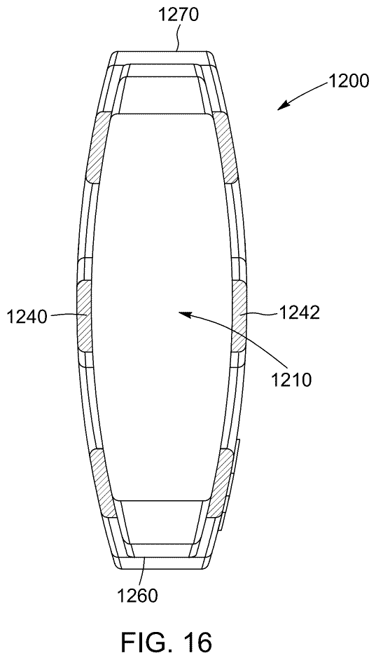

As best shown in FIG. 16, the first and second side walls 1240, 1242 form first and second concavities facing each other and forming—or delimiting—at least partially the hollow space.

In the embodiment shown, each of the first and second connecting portions 1260, 1270 comprises at least one gripping part. For instance, each of the first and second connecting portions comprises first and second gripping parts spaced apart from each other and forming apexes of the corresponding one of the first and second connecting portions. For instance, a concavity is formed in gripping edges of the first and second sidewalls 1240, 1242, between the first and second gripping parts of the corresponding one of the first and second connecting portions.

In the embodiment shown, the tip 1321 of one of the user-contacting curved end portions extends beyond the apexes formed by the first and second gripping parts of the corresponding one of the first and second connecting portions 1260, 1270. In other words, when the urinary device 1100 is supported on a support-contacting surface with the second user-contacting end portion 1320 being closer to the support-contacting surface than the first user-contacting end portion 1310, the tip 1321 of the user-contacting end portion 1320 contacts the support-contacting surface.

It is appreciated that the shape and the configuration of second embodiment of the urinary device, for instance the shape and configuration of the side walls, the sealing rim, the urine-collecting body and the first and second connecting portions can vary from the embodiment shown.

### Reversible Female Urinary Assembly

The present disclosure also concerns a reversible female urinary assembly, comprising a reversible female urinary device 100, 1100 according to the present disclosure and a disposable bag fittable into the hollow space and securable to the body. The urinal device 100, 1100 is indeed designed for use with disposable hygienic bags which are shaped to fit the particular design of the urinary device 100, 1100 and to be removably securable thereto. Typically, a body portion of a hygienic bag is inserted into the hollow space 210, 1210 of the body 200, 1200 (or urine-collecting housing 200, 1200), for instance via one of the lateral holes or via one of the bag-introducing openings. An open end of the disposable hygienic bag is then secured to the body, while covering at least partially one of the first and second sealing rims 300, 400, 1300, depending on the configuration chosen by the user. If the bag comprises a closure string slidably mounted to the opening thereof, the closure string can be used to tie the opening of the bag at a junction of the corresponding one of the first and second sealing rims and the body 200, 1200. Alternatively, the bag can comprise an elastic band which secures the bag to the body of the urinary device 100, 1100 by applying pressure to the outer surface of the body 200, 1200, proximate the corresponding one of the first and second longitudinal ends 220, 230, 1221, 1231.

The hygienic bag might further comprise an absorbent material layer, such as a gel, which captures liquids. The absorbent material layer substantially instantaneously holds liquids to ease handling of the urinary assembly. The absorbent material layer can be a distinct component from the bag or it can be provided as a single piece.

It is thus understood that the female urinary device 100 can be used when the user is sitting or lying and is shaped and dimensioned to substantially conform to a plurality of morphologies.

As mentioned above, the first and second user-contacting surfaces 302, 402, 1302 formed by the first and second sealing rims 300, 400, 1300 are shaped and dimensioned to come in contact with the user, and more particularly with the area surrounding their genitals. The urinary device 100, 1100 is thus adapted to different morphologies due to the specific shapes and dimensions of the first and second sealing rims 300, 400, 1300 and to the specific shapes, dimensions and relative arrangement of the first and second user-contacting curved portions of the first and second sealing rims 300, 400, 1300.

It is understood that the position of the urinary device 100, 1100 between the legs of the user can easily be adjusted by gripping any one of the first and second gripping parts of the first and second connecting portions to better adjust the position of the selected one of the first and second sealing rims. As mentioned above, the urinary device 100, 1100 is thus configurable into distinct configurations. As detailed above, the first embodiment of the urinary device 100 is configurable into four usage configurations: a first configuration, wherein the first connecting portion 260 is supported on a supporting surface and the first sealing rim 300 is in contact with or proximate the genitals of the user (arrow C1 on FIG. 4), the first user-contacting curved portion 310 of the first sealing rim 300 bordering a lower portion of the genitals of the user; a second configuration, wherein the first connecting portion 260 is supported on a supporting surface and the second sealing rim 400 is in contact with or proximate the genitals of the user (arrow C2 on FIG. 4), the first user-contacting curved portion 410 of the second sealing rim 400 bordering a lower portion of the genitals of the user; a third configuration, wherein the second connecting portion 270 is supported on a supporting surface and the first sealing rim 300 is in contact with or proximate the genitals of the user (arrow C3 on FIG. 5), the second user-contacting curved portion 320 of the first sealing rim 300 bordering a lower portion of the genitals of the user; and a fourth configuration, wherein the second connecting portion 270 is supported on a supporting surface and the second sealing rim 400 is in contact with or proximate the genitals of the user (arrow C4 on FIG. 5), the second user-contacting curved portion 420 of the second sealing rim 400 bordering a lower portion of the genitals of the user. As detailed above, the second embodiment of the urinary device 1100, which comprises one single sealing rim, is configurable into two distinct usage configurations.

It is also understood that the female urinary device 100, 1100 is shaped and dimensioned to support a substantially filled bag arranged in the hollow space 210, 1210 and is easily cleanable. For instance, the urinary device is at least partially made of plastic, rubber or any suitable material having flexibility and resilience properties, that can be easily cleanable and/or that provides a substantially smooth surface, in order to increase the comfort of use.

Several alternative embodiments and examples have been described and illustrated herein. The embodiments of the disclosure described above are intended to be exemplary only. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. It is understood that the disclosure may be embodied in other specific forms without departing from the central characteristics thereof. The present examples and embodiments, therefore, are to be considered in all respects as illustrative and not restrictive, and the disclosure is not to be limited to the details given herein. Accordingly, while the specific embodiments have been illustrated and described, numerous modifications come to mind. The scope of the disclosure is therefore intended to be limited by the scope of the appended claims.

1-56. (canceled)

57. A reversible female urinary device comprising:
a urine-collecting housing defining a hollow space and having at least one urine-collecting inlet opening into the hollow space and having an inlet height; and
at least one sealing rim at least partially surrounding the urine-collecting inlet,
wherein, considered along the inlet height, the sealing rim forms first and second opposed user-contacting curved end portions, thereby defining at least two distinct usage configurations of the urinary device.

58. The urinary device according to claim 57,
wherein the urinary device has a longitudinal direction,
wherein the urine-collecting housing comprises first and second opposed longitudinal ends,
wherein the urine-collecting inlet is formed at one of the first and second longitudinal ends, and
wherein the sealing rim surrounds substantially an entirety of a periphery of the urine-collecting inlet.

59. The urinary device according to claim 58,
wherein, considered in a plane comprising the longitudinal direction and the inlet height, the sealing rim has a curvature that varies along the inlet height.

60. The urinary device according to claim 58,
wherein tips of the first and second user-contacting curved portions of the sealing rim are longitudinally offset with respect to each other.

61. The urinary device according to claim 58,
wherein the urine-collecting housing has a bag-introducing opening formed at the other one of the first and second longitudinal ends, opening into the hollow space, and
wherein the other one of the first and second longitudinal ends forms a handling portion shaped and dimensioned to increase a contact of the sealing rim with the user when in use.

62. The urinary device according to claim 58,
wherein the at least one urine-collecting inlet comprises first and second urine-collecting inlets having first and second inlet heights, opening into the hollow space and formed respectively at the first and second longitudinal ends,
wherein the at least one sealing rim comprises first and second sealing rims at least partially surrounding respectively the first and second urine-collecting inlets,
wherein, considered along the corresponding one of the first and second inlet heights, each of the first and second sealing rims forms first and second opposed user-contacting curved end portions, thereby defining at least four distinct usage configurations of the urinary device,
wherein the first and second sealing rims surround respectively substantially an entirety of a periphery of the first and second urine-collecting inlets, and
wherein, considered in a plane comprising the longitudinal direction and the first and second inlet heights, the first and second user-contacting curved portions of each of the first and second sealing rims have first and second radii of curvature different from each other.

63. The urinary device according to claim 57,
wherein the urine-collecting housing comprises first and second side walls spaced apart from each other, and first and second connecting portions extending at least partially between the first and second side walls,
wherein gripping openings are formed in the first and second side walls, substantially in register with each other, and
wherein a periphery of each gripping opening forms a convexity substantially following a profile of a closest edge of the corresponding one of the first and second side walls.

64. The urinary device according to claim 63,
wherein the first and second side walls form respectively first and second concavities facing each other and forming at least partially the hollow space.

65. The urinary device according to claim 63,
wherein at least one of the first and second connecting portions comprises first and second gripping parts spaced apart from each other and forming apexes of the corresponding one of the first and second connecting portions, and
wherein first and second concavities are formed in gripping edges of the first and second side walls, between the first and second gripping parts of the corresponding one of the first and second connecting portions.

66. The urinary device according to claim 57,
wherein the urinary device is at least partially made of plastic and is at least partially injection-molded.

67. A reversible female urinary device having a longitudinal direction and comprising:
a body at least partially delimiting a hollow space and having first and second opposed longitudinal ends, at least one urine-collecting inlet being formed at one of the first and second longitudinal ends, the urine-collecting inlet opening into the hollow space and having an inlet height; and
a sealing flange extending outward and away from the urine-collecting inlet,
wherein, considered along the inlet height, the sealing flange forms first and second opposed user-contacting curved portions, thereby defining at least two possible usage configurations of the urinary device.

68. The urinary device according to claim 67,
wherein, considered in a plane comprising the longitudinal direction and the inlet height, the first and second user-contacting curved portions have first and second radii of curvature different from each other.

69. The urinary device according to claim 68,
wherein the plane comprising the longitudinal direction and the inlet height forms a plane of symmetry of the urinary device.

70. The urinary device according to claim 67,
wherein tips of the first and second user-contacting curved portions of the sealing flange are longitudinally offset with respect to each other.

71. The urinary device according to claim 70,
wherein the sealing flange is formed integral with the body.

72. The urinary device according to claim 67,
wherein the at least one urine-collecting inlet comprises first and second urine-collecting inlets having first and second inlet heights, opening into the hollow space and formed respectively at the first and second longitudinal ends,
wherein the at least one sealing flange comprises first and second sealing flanges at least partially surrounding respectively the first and second urine-collecting inlets,
wherein, considered along the corresponding one of the first and second inlet heights, each of the first and second sealing flanges forms first and second opposed user-contacting curved end portions, thereby defining at least four distinct usage configurations of the urinary device, and
wherein the first and second sealing flanges surround respectively substantially an entirety of a periphery of the first and second urine-collecting inlets.

73. The urinary device according to claim 72,
wherein, considered in a plane comprising the longitudinal direction and the first and second inlet heights, the first and second user-contacting curved portions of each of the first and second sealing flanges have first and second radii of curvature different from each other, and
wherein the plane comprising the longitudinal direction and the first and second inlet heights forms a plane of symmetry of the urinary device.

74. The urinary device according to claim 67,
wherein the body comprises first and second side walls spaced apart from each other, and first and second connecting portions extending at least partially between the first and second side walls,
wherein first and second gripping openings are formed in at least one of the first and second side walls, and
wherein a periphery of the gripping opening forms a convexity substantially following a profile of a closest edge of the corresponding one of the first and second side walls.

75. The urinary device according to claim 67,
wherein the urinary device is single-pieced.

76. A reversible female urinary assembly, comprising:
a reversible female urinary device according to claim 57; and
a disposable bag fittable into the hollow space and securable to the urine-collecting housing.
